(19) United States
(12) Patent Application Publication
(10) Pub. No.: US 2014/0121642 A1
(43) Pub. Date: May 1, 2014

(54) DUAL FUNCTION MEDICAL DEVICES

(71) Applicant: Boston Scientific Scimed, Inc., Maple Grove, MN (US)

(72) Inventors: Gary A. Jordan, Litchfield, NH (US); John A. Griego, Blackstone, MA (US); Matthew B. Hollyer, Williamstown, VT (US)

(73) Assignee: Boston Scientific Scimed, Inc., Maple Grove, MN (US)

(21) Appl. No.: 14/054,374

(22) Filed: Oct. 15, 2013

Related U.S. Application Data
(60) Provisional application No. 61/718,402, filed on Oct. 25, 2012.

(51) Int. Cl. A61M 25/09

(52) U.S. Cl. CPC A61M 25/09
USPC 604/510; 604/528

(57) ABSTRACT

Medical devices and methods for making and using medical devices are disclosed. An example medical device may include a dual function medical device. The dual function medical device may include an inner guidewire. An outer guidewire may be disposed about the inner guidewire. The outer guidewire may include a tubular member having a plurality of slots formed therein. A torque member may be coupled to the dual function medical device. The torque member may be configured to independently attach to the inner guidewire and the outer guidewire.

### CROSS-REFERENCE TO RELATED APPLICATIONS

This application claims the benefit under 35 USC §119 of U.S. Provisional Application No. 61/718,402, filed on Oct. 25, 2012, the entirety of which is incorporated herein by reference.

### TECHNICAL FIELD

The present disclosure pertains to medical devices, and methods for manufacturing medical devices. More particularly, the present disclosure pertains to dual function guidewire that include an inner guidewire and an outer guidewire.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An example medical device may include a dual function medical device. The dual function medical device may include an inner guidewire. An outer guidewire may be disposed about the inner guidewire. The outer guidewire may include a tubular member having a plurality of slots formed therein. A torque member may be coupled to the dual function medical device. The torque member may be configured to independently attach to the inner guidewire and the outer guidewire.

Methods for accessing a region of the biliary tree are also disclosed. An example method may include providing a dual function guidewire. The dual function guidewire may include an inner guidewire and an outer guidewire disposed about the inner guidewire. The outer guidewire may include a tubular member having a plurality of slots formed therein. The method may also include advancing the dual function guidewire through a body lumen to a position adjacent to an area of interest, and either advancing a first medical device over the outer guidewire to the area of interest or removing the outer guidewire from the inner guidewire and advancing a second medical device over the inner guidewire to the area of interest.

Dual function guidewires for accessing a body lumen along the biliary tree of a patient are also disclosed. An example dual function guidewire may include an inner guidewire. The inner guidewire may have first outer diameter that is configured to guide a first medical device to an area of interest. An outer guidewire may be disposed about the inner guidewire. The outer guidewire may have a second outer diameter that is configured to guide a second medical device to the area of interest. The inner guidewire may have a pre-formed bend formed therein. The outer guidewire may include a tubular member having a plurality of slots formed therein. The first outer diameter may closely approximate an inner diameter of the outer guidewire.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is an overview of the biliary tree;

FIG. 2 is a cross-sectional side view of a portion of an example inner guidewire;

FIG. 3 is a side view of a portion of an example outer guidewire;

FIG. 3A is a side view of a portion of an example outer guidewire with a pre-formed bend;

FIG. 3B is a side view of a portion of an example outer guidewire in a loop configuration;

FIGS. 4-7 are side views illustrating a number of different configurations for an example dual function guidewire;

FIG. 8 is a cross-sectional view of an example torque member for use with a dual function guidewire;

FIG. 9 is a partially cross-sectional side view of a medical device in use with a dual function guidewire;

FIG. 10 is a partially cross-sectional side view of another example medical device in use with an example inner guidewire;

FIG. 11 is a side view of another example dual function guidewire;

FIG. 12 is a cross-sectional view taken through line 12-12 in FIG. 11;

FIG. 13 is a cross-sectional view of an example dual function guidewire in a second configuration;

FIG. 14 is a cross-sectional view of an example dual function guidewire in a first configuration; and

FIG. 15 is a cross-sectional view of an example dual function guidewire in a second configuration.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Endoscopic retrograde cholangiopancreatography (ERCP) is used primarily to diagnose and treat conditions of the bile ducts including, for example, gallstones, inflammatory strictures, leaks (e.g., from trauma, surgery, etc.), and cancer. Through the endoscope, the physician can see the inside of the stomach and duodenum, and inject dyes into the ducts in the biliary tree and pancreatic tract so they can be seen on x-rays. These procedures may necessitate gaining and keeping access to the biliary duct, which may be technically challenging, may require extensive training and practice to gain proficiency, and may require one or more expensive tools in order to perform.

During an ERCP procedure, a number of steps are typically performed while the patient is often sedated or anaesthetized. For example, an endoscope may be inserted through the mouth, down the esophagus, into the stomach, through the pylorus into the duodenum, to a position at or near the ampulla of Vater (the opening of the common bile duct and pancreatic duct). Due to the shape of the ampulla and the angle at which the common bile and pancreatic ducts meet the wall of the duodenum, the distal end of the endoscope is generally placed just past the ampulla. Due to the positioning of the endoscope beyond the ampulla, the endoscopes used in these procedures are usually side-viewing endoscopes. The side-viewing feature provides imaging along the lateral aspect of the tip rather than from the end of the endoscope. This allows the clinician to obtain an image of the medial wall of the duodenum, where the ampulla of Vater is located, even though the distal tip of the endoscope is beyond the opening.

Next, a clinician may cannulate the entrance to the pancreatic and bile ducts, which are located beyond the ampulla of Vater, with a guidewire, catheter, or cannula placed through the instrument channel of the endoscope. The devices may be directed cranially at an angle with respect to the distal end of the endoscope, so as to facilitate insertion into the opening. Once in place within the ampulla, a radiocontrast agent can be injected into the bile ducts and/or pancreatic duct. Fluoroscopy can then be used to identify and treat various ailments, including blockages or leakage of bile into the peritoneum (abdominal cavity).

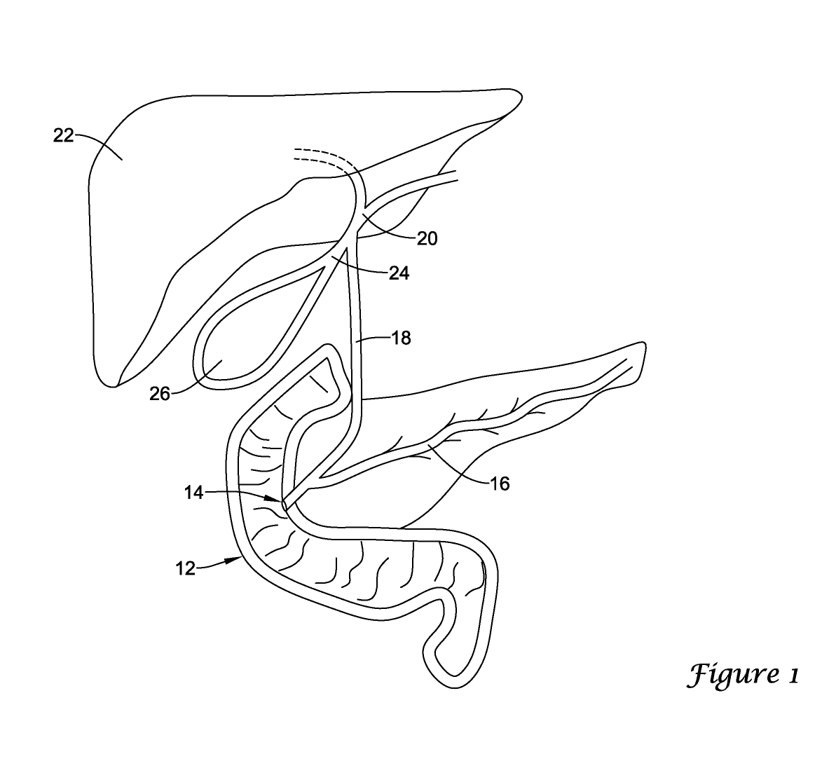

FIG. 1 provides an overview of the biliary system or tree and/or pancreas or pancreatic tract. Illustrated is a portion of the duodenum 12 where the ampulla of Vater 14 is located. For the purposes of this disclosure, the ampulla of Vater 14 is understood to be the same anatomical structure as the papilla of Vater. The ampulla of Vater 14 generally forms the opening where the pancreatic duct 16 and the bile duct 18 can empty into the duodenum 12. The hepatic ducts, generally bearing reference number 20, are connected to the liver 22 and empty into the bile duct 18. Likewise, the cystic duct 24, which is connected to the gall bladder 26, also empties into the bile duct 18. In general, an endoscopic or biliary procedure may include advancing a medical device to a suitable location along the biliary tree and/or pancreatic tract and then performing the appropriate intervention.

Because the ampulla of Vater 14 is positioned within the duodenum 12, and because the duodenum 12 may be moving due to peristalsis, positioning and cannulating the ampulla of Vater 14 may be challenging. Other factors may also complicate cannulating the biliary tree and/or the pancreatic tract as well as complicate maintaining access to various portions of the biliary tree and/or the pancreatic tract. Disclosed herein are systems, tools, and methods for cannulating the ampulla of Vater during the diagnosis and treatment of biliary, hepatic, gallbladder, and/or pancreatic disease or other ailments. The systems, tools, and methods disclosed are generally directed at improving the ability of a user to cannulate and maintain access to the appropriate target region along the biliary tree and/or the pancreatic tract during an intervention.

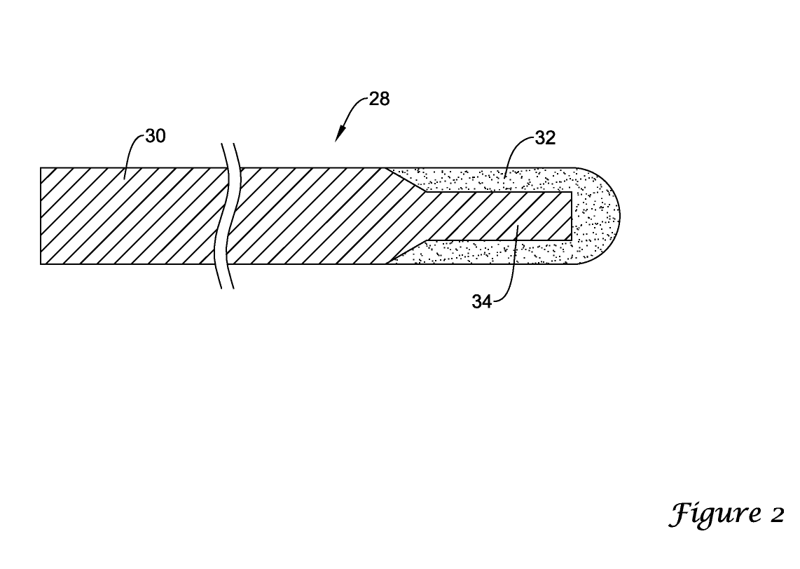

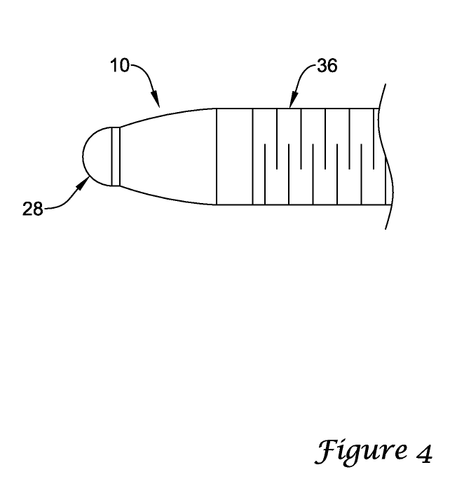

FIG. 2 illustrates a portion of an example dual function guidewire (e.g., dual function guidewire 10 as shown in FIGS. 4-10) that may improve the ability of a user to cannulate and maintain access to regions along the biliary tree and/or the pancreatic tract. For example, a dual function guidewire may include a first or “inner” guidewire 28. As shown schematically in FIG. 2, inner guidewire 28 may include a core wire or member 30 and a tip member 32 coupled to core member 30. In at least some embodiments, tip member 32 may include a polymer layer disposed over core member 30. Alternatively, tip member 32 may include a coil or “spring tip”. These are just examples. Other configurations are contemplated.

In general, inner guidewire 28 is configured to guide relatively “smaller” treatment devices to the appropriate location along the biliary tree and/or pancreatic tract. For example, inner guidewire 28 may have an outer diameter of about 0.010 to 0.040 inches, or about 0.012 to 0.035 inches, or about 0.014 to 0.030 inches, or about 0.025 inches. These are just examples. Other sizes are contemplated. A treatment device passed over inner guidewire 28 would generally have a lumen formed therein that is sized to fit over inner guidewire 28. In at least some embodiments, the treatment device may be deigned to work with a “smaller” guidewire of the size of inner guidewire 28. Some example devices may include stent (e.g., drainage stent) delivery systems, tomes or cutting devices (e.g., sphincterotomes), catheters, and the like.

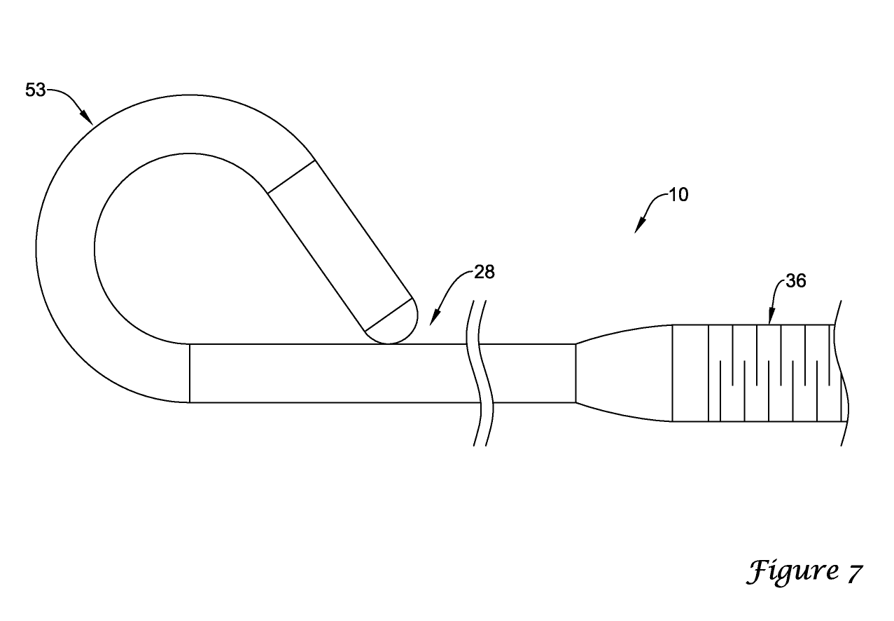

A portion of core member 30 may taper or narrow. In at least some embodiment, core member 30 may include a barrel or constant diameter region 34. Barrel region 34 may be positioned adjacent to the distal end of core member 30 or at a location that is proximal of the distal end of core member 30. Barrel region 34 may be disposed along or otherwise define a region of inner guidewire 28 that is configured to buckle or loop back upon itself (see, for example, FIG. 7, which illustrates inner guidewire 28 in a looped configuration).

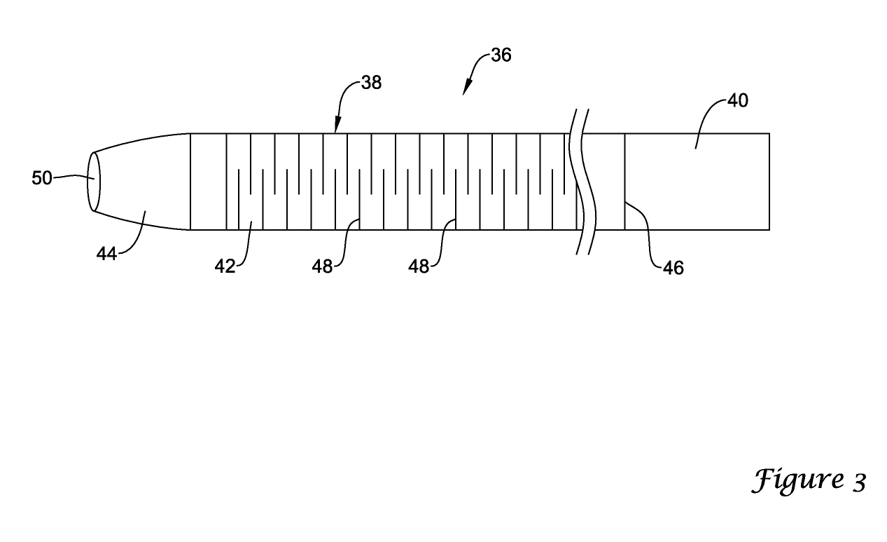

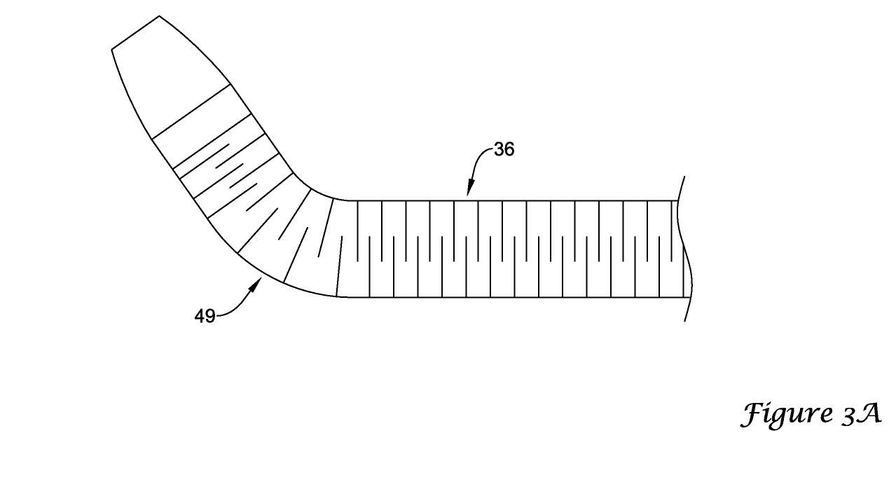

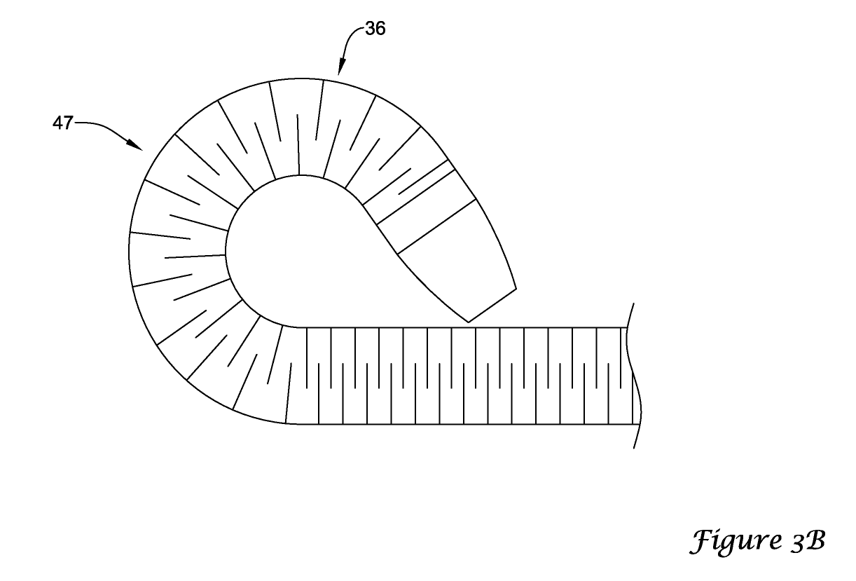

FIG. 3 illustrates another component of a dual function guidewire. Here, an example outer guidewire 36 can be seen. Outer guidewire 36 may include a tubular member or body 38 having a proximal region 40, a distal region 42, and a tip region 44. In general, outer guidewire 36 may be configured to be disposed about inner guidewire 28 (see, for example, FIGS. 4-7). In general, outer guidewire 36 is configured to guide relatively “larger” treatment devices to the appropriate location along the biliary tree and/or pancreatic tract. For example, outer guidewire 36 may have an outer diameter of about 0.020 to 0.045 inches, or about 0.025 to 0.040 inches, or about 0.030 to 0.035 inches, or about 0.035 inches. These are just examples. Other sizes are contemplated. A treatment device passed over outer guidewire 36 would generally have a lumen formed therein that is sized to fit over outer guidewire 36. In at least some embodiments, the treatment device may be deigned to work with a “larger” guidewire of the size of outer guidewire 36. Some example devices may include stent (e.g., drainage stent) delivery systems, tomes or cutting devices (e.g., sphincterotomes), catheters, and the like.

In at least some embodiments, outer guidewire 36 may have an inner diameter that approximates the outer diameter of inner guidewire 28. Accordingly, a relatively tight fit between inner guidewire 28 and outer guidewire 36 may allow the relative positions of guidewire 28/36 to be maintained relative to one another. In other embodiments, some spacing may be incorporated by enlarging the inner diameter of outer guidewire 36 relative to the outer diameter of inner guidewire 28 so that outer guidewire 36 and inner guidewire 28 may be more freely movable with respect to one another.

Outer guidewire 36 may include a number of additional structural features and/or configurations. For example, in some embodiments proximal region 40, distal region 42, and tip region 44 may be formed from the same structure (e.g., tubular member 38). Alternatively, two or more of proximal region 40, distal region 42, and tip region 44 may be formed from separate structures that are secured together. For example, proximal region 40 and distal region 42 may be formed from different tubular members that are joined at a joint 46. In at least some of these embodiments, distal region 42 may be formed from a first material (e.g., a nickel-titanium alloy) and proximal region 40 may be formed from a second, different material (e.g., a nickel-chromium-molybdenum alloy). These are just examples. Regions 40/42 may be joined together with a suitable bond or connector. Some examples of suitable configurations and/or structures can be utilized for joining regions 40/42 may include those described in U.S. Pat. Nos. 6,918,882 and 7,071,197 and/or in U.S. Patent Pub. No. 2006-0122537, the entire disclosures of which are herein incorporated by reference.

Distal region 42 may have a plurality of slots 48 formed therein. Slots 48 may provide tubular member 38 with increased flexibility while also maintaining the ability to transmit torque along the length of tubular member 38. A variety of different patterns and/or configurations are contemplated for slots 48. Some of these variations are disclosed herein. In some embodiments, distal region 42 and/or other portions of tubular member 38 may have a liner disposed along an inner surface, an outer surface, or both thereof. This may include a liner that “seals” slots 48. In other embodiments, distal region 42 and/or other portions of tubular member 38 may be substantially free of a liner disposed along an inner surface, an outer surface, or both thereof. In some of these and in other embodiments, distal region 42 and/or other portions of tubular member 38 may take the form of a “bare metal” tube.

Tubular member 38 may have a lumen 50 formed therein. In general, lumen 50 may be configured to receive inner guidewire 28 as shown in FIG. 4, thereby forming an assembly therewith and defining dual function guidewire 10. With inner guidewire 28 disposed within outer guidewire 36, dual function guidewire 10 may be advanced through a body lumen to a position adjacent a target region, for example along the biliary tree. While advancing dual function guidewire 10 through the anatomy, a number of different configurations may be utilized. For example, inner guidewire 28 may be positioned so that only a relatively short tip portion thereof may extend from the distal end of outer guidewire 36. Such a configuration may provide an atraumatic surface, which may be desirable during navigation. In such a configuration, dual function guidewire 10 may be advanced through the anatomy. This may include disposing an endoscope (not shown) within a portion of the anatomy and advancing dual function guidewire 10 through the endoscope.

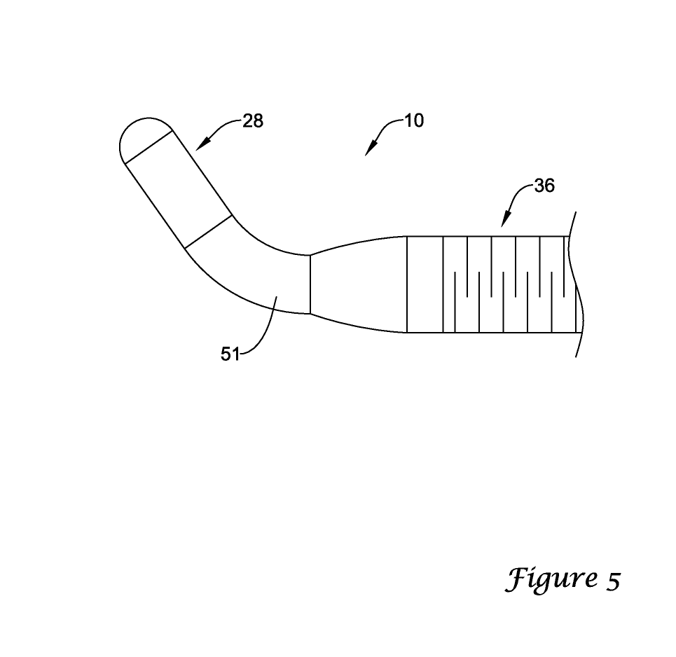

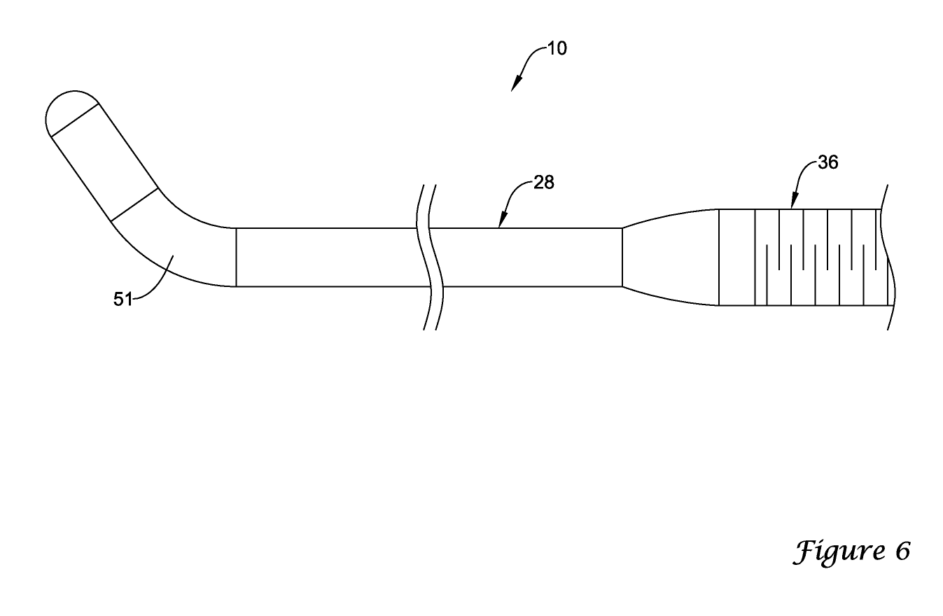

Outer guidewire 36 may include a number of additional structural features. For example, outer guidewire 36 may include one or more pre-formed bends including pre-formed bend 49 as shown in FIG. 3A. In addition, outer guidewire 36 may be configured to buckle or otherwise fold back upon itself and define a loop region 47 as shown in FIG. 3B. In addition, outer guidewire 36 may also include a suitable connector (e.g., a tuohy-borst connector, Y-connenctor, or the like) that allows contrast fluid or other fluids to be delivered through outer guidewire 36. Therefore, outer guidewire 36 may be utilized for fluid delivery to target regions during an intervention. Other variations are also contemplated for outer guidewire 36. In some embodiments, inner guidewire 28 may be shifted (e.g., moved distally) relative to outer guidewire 36 as shown in FIG. 5. For example, inner guidewire 28 may include a pre-formed bend 51 and only the portion of inner guidewire 28 extending distally from pre-formed bend 51 may extend from outer guidewire 36. Alternatively, a larger portion of inner guidewire 28 may extend from outer guidewire 36 as shown in FIG. 6. This may allow dual function guidewire 10 to have a curved or bent tip, which may aid in navigation of dual function guidewire 10 through the anatomy.

As alluded to herein, inner guidewire 28 may be configured to buckle or otherwise fold back upon itself and define a loop region 53 as shown in FIG. 7. For example, inner guidewire 28 may be configured to shift between a first configuration and a looped configuration. In at least some embodiments, inner guidewire 28 may shift to the looped configuration when encountering a tightening or stricture in the anatomy. While in the looped configuration, inner guidewire 28 may be able to pass through the stricture. This may include distally advancing inner guidewire 28 (while keeping outer guidewire 36 substantially stationary) through the stricture and then passing outer guidewire 36 over inner guidewire 28 through the stricture. Alternatively, only inner guidewire 28 may be passed through the stricture and, if desired, a suitable medical device can be advanced over inner guidewire 28 through the stricture.

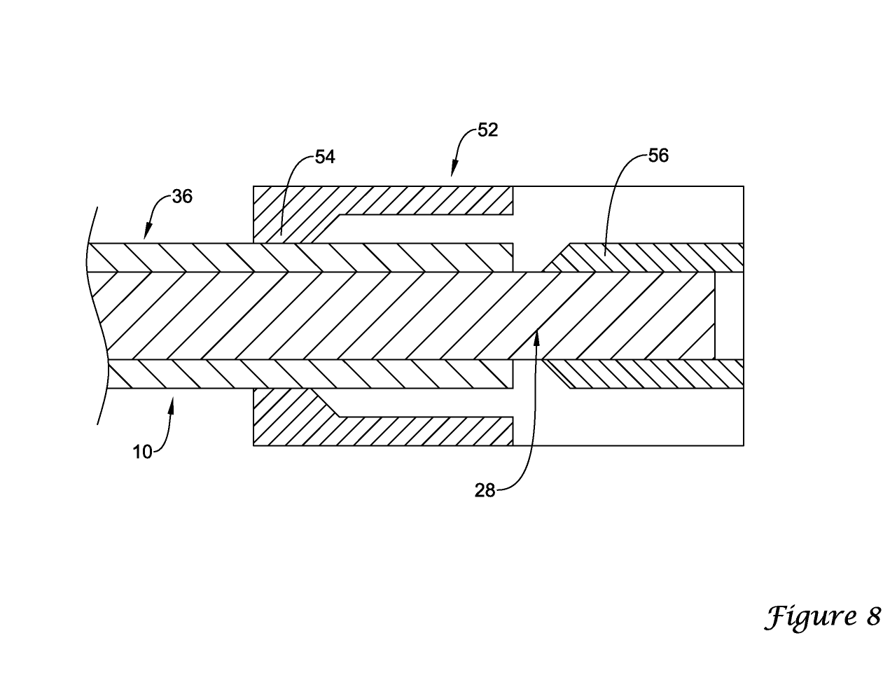

FIG. 8 shows schematically that a locking or torque member 52 may be secured to dual function guidewire 10. For example, torque member 52 may include a first lock or collet member 54 that is configured to secure to outer guidewire 36. Torque member 52 may also include a second lock or collet member 56 that is configured to secure to inner guidewire 28. In general, torque member 52 may be configured to secure the position of one or both of guidewire 28/36. In other words, inner guidewire 28, outer guidewire 36, or both may be independently secured to torque member 52. This may allow a user to manipulate the position of one of guidewire 28/36 while maintaining the position of the other guidewire 28/36. In addition to providing locking features, torque member 52 may also be utilized to rotate dual function guidewire 10 (and/or one of inner guidewire 28 and outer guidewire 36). The precise form of torque member 56 may vary to include a variety of different structures.

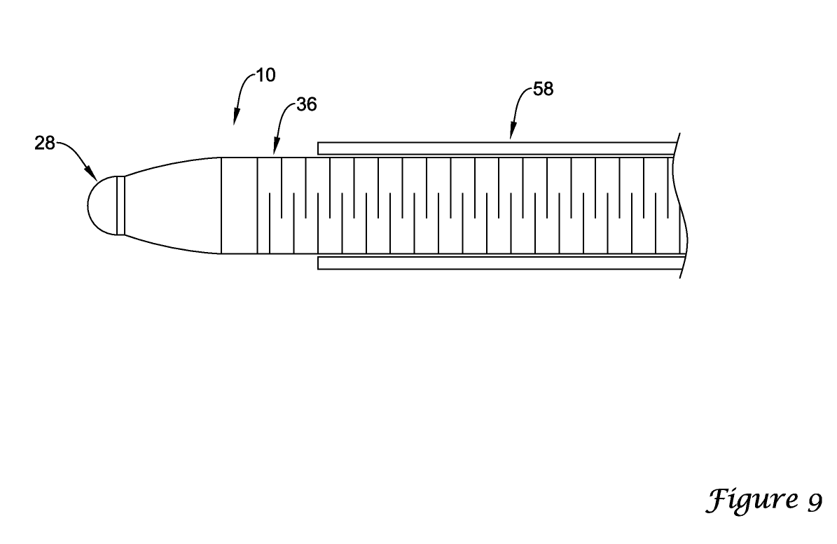

In use, dual function guidewire 10 may be advanced through a body lumen to a position adjacent to an area of interest. Once positioned, a number of different medical devices may be utilized to treat the patient. Depending on the intervention, the particular medical device utilized for treatment may vary. For example, as indicated above some interventions may utilize a “larger” medical device 58 (shown schematically) as shown in FIG. 9. In general, medical device 58 defines a lumen sufficient for it to track over dual function guidewire 10 (e.g., over outer guidewire 36). Thus, dual function guidewire 10 may be advanced through the anatomy to a suitable position adjacent to a target region and medical device 58 may be advanced over dual function guidewire 10 (e.g., over outer guidewire 36). When properly positioned, medical device 58 may be utilized to perform the desired treatment intervention.

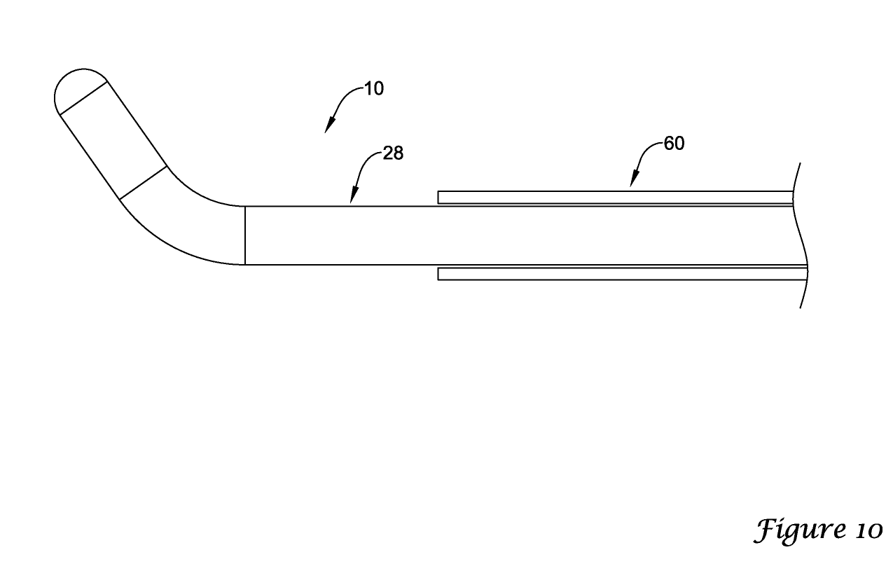

Alternatively, the desired intervention may utilize a “smaller” medical device 60 (shown schematically) as illustrated in FIG. 10. In general, medical device 60 defines a lumen sufficient for it to track over inner guidewire 28. Thus, advancing medical device 60 to the target region may include removing outer guidewire 36 from inner guidewire 28 and then advancing medical device 60 over inner guidewire 28. As alluded to herein, the form of medical device 58/60 can vary and may include stent (e.g., drainage stent) delivery systems, tomes or cutting devices (e.g., sphincterotomes), catheters, and the like. Because dual function guidewire 10 can be utilized to delivery both “larger” devices (e.g., medical device 58) and “smaller” devices (e.g., medical device 60) without having to remove the entire dual function guidewire 10, dual function guidewire 10 may desirably aid in maintaining access to the anatomy during an intervention. In other words, the use of dual function guidewire 10 may allow a clinician to guide a variety of differently sized medical devices to a target region without having to remove a previously-placed guidewire and then regain access to the anatomy. This may reduce the total number of step needed to perform an intervention, reduce the total time for the intervention, and generally simplify the intervention.

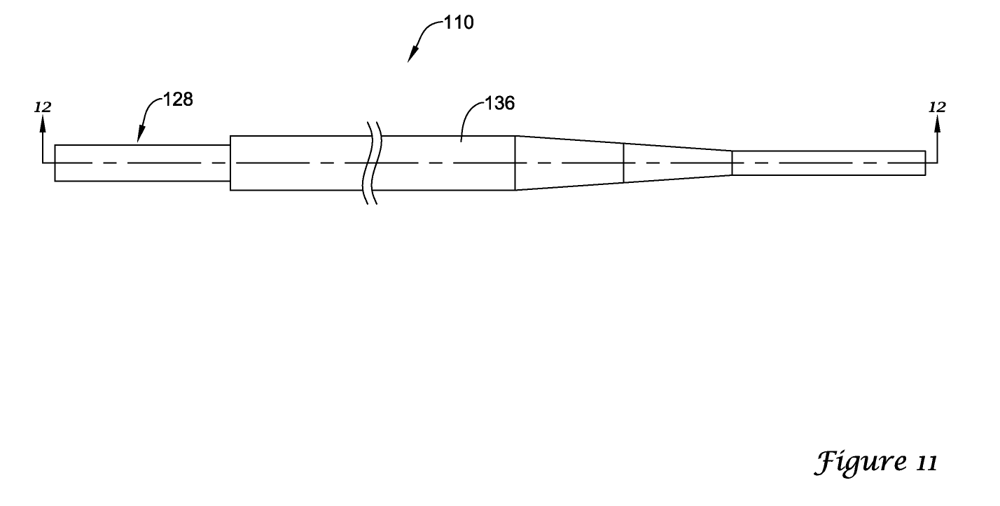

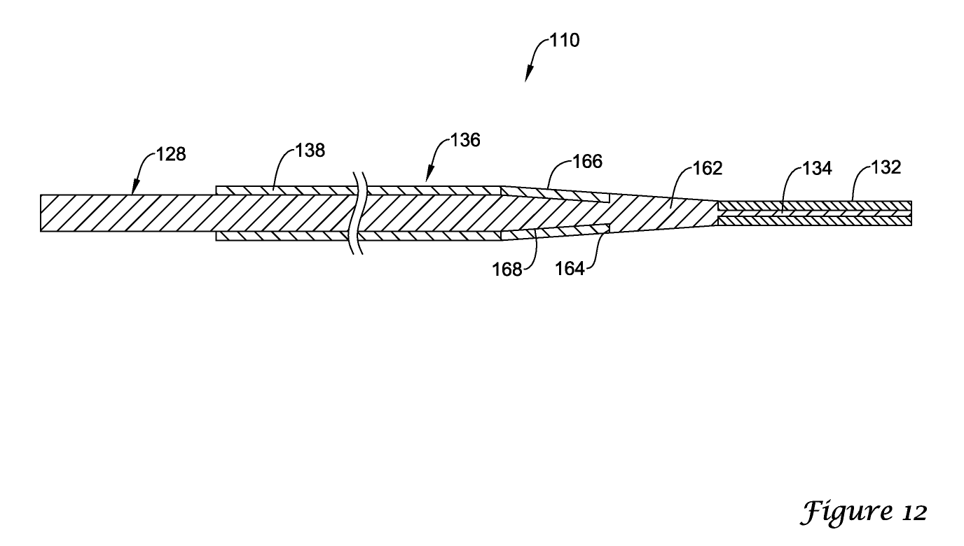

FIG. 11 illustrates another example dual function guidewire 110 that may be similar in form and function to other dual function guidewires disclosed herein. Dual function guidewire 110 may include inner guidewire 128 and outer guidewire 136. As shown in FIG. 12, inner guidewire 128 may include a distal region 162 defining a ridge or shoulder 164. Inner guidewire 128 may also have tip region 134 and tip member 132. The precise form of tip region 134 and tip member 132 may vary. In some embodiments, these portions may have a form similar to inner guidewire 28 or another suitable form. In addition, dual function guidewire 110 may be used with a torque member similar to torque member 52 disclosed herein.

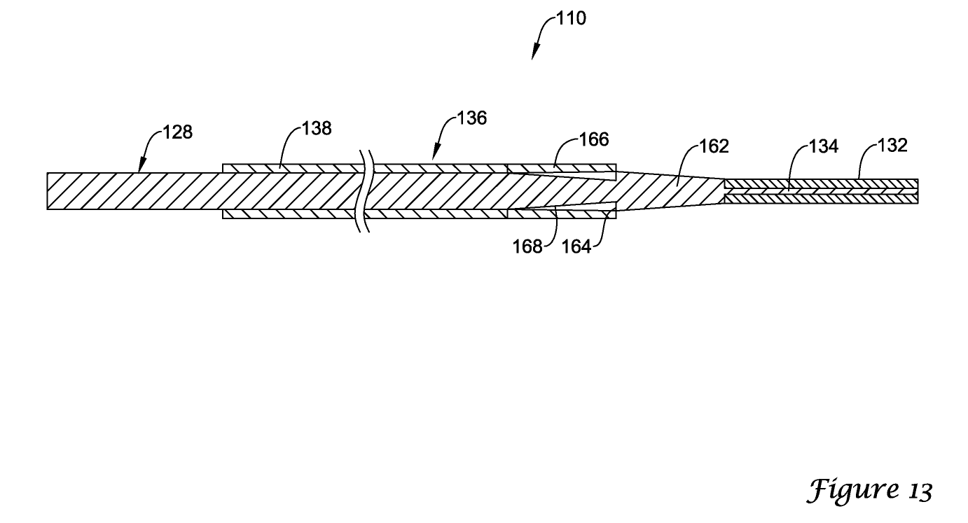

Outer guidewire 136 may include tubular member 138 having a distal collet or deflectable region 166. In general, deflectable region 166 may be configured to shift between a first or “locked” configuration and a second or “deflected” configuration as shown in FIG. 13. When in the first configuration, deflectable region 166 may engage shoulder 164 inner guidewire 128. This may aid in transferring pushing force applied to outer guidewire 136 to inner guidewire 128 and/or otherwise aid in navigating dual function guidewire 110 through the anatomy. When shifted to the second configuration, deflectable region 166 may “disengage” from shoulder 164 so that outer guidewire 136 may be advanced distally past shoulder 164.

In at least some embodiments, deflectable region 166 may be formed from a shape memory material. Thus, exposure to an appropriate stimulus may cause deflectable region 166 to shift between configurations. For example, deflectable region 166 may be configured to shift between configurations when exposed to a particular temperature. This may include shifting to one of the first or the second configurations when exposed to a temperature approximating body temperature or another temperature. In other embodiments, electrical current may be used to change the temperature or otherwise cause deflectable region 166 to shift between configurations. For example, deflectable region 166 may be shifted to one of the first or second configurations when current is applied to outer guidewire 136.

In one example, deflectable region 166 may be configured to be in the first configuration. As outer guidewire 136 is advanced over inner guidewire 128, the outer surface or diameter of inner guidewire 128 may cause deflectable region 166 to deflect radially outward (e.g., cause deflectable region 166 to shift to the deflected configuration). As outer guidewire 136 is further advanced to a ground region 168 of inner guidewire 128, deflectable region 166 may track along ground region 168 and begin to shift radially inward and, ultimately, shift to the first configuration. In the first configuration, deflectable region 166 may abut shoulder 164 so that pushing forces may be transferred from outer guidewire 136 to inner guidewire 128.

In some of these and in other embodiments, one or more structural features may be utilized to shift deflectable region 166 between configurations. For example, outer guidewire 136 may include one or more longitudinal slots that allow for a certain amount of deflectability in deflectable region 166. For example, the longitudinal slots may allow deflectable region 166 to deflect when a certain amount of force is applied to outer guidewire 136.

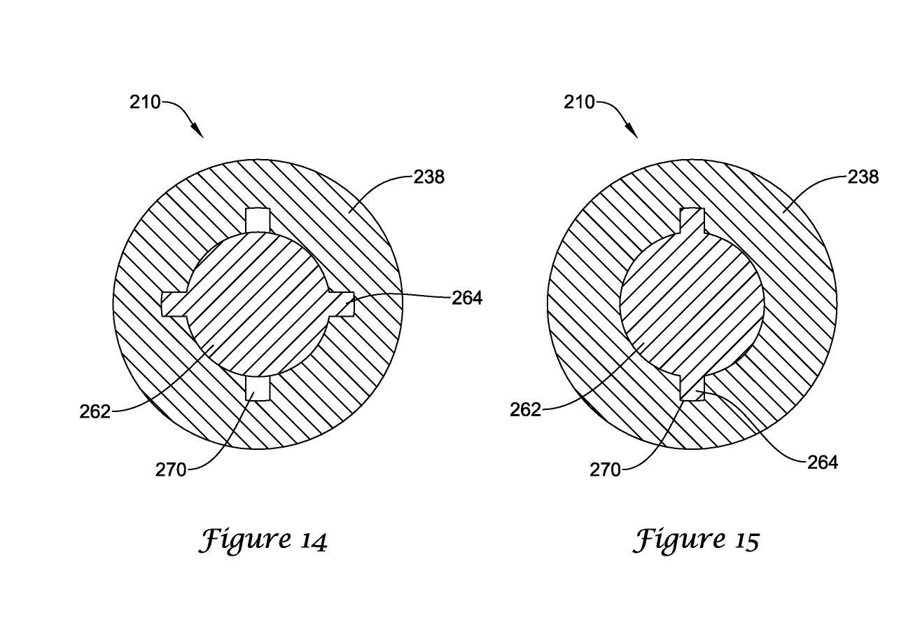

FIGS. 14-15 illustrate a portion of dual function guidewire 210 that may be similar in form and function to other dual function guidewires disclosed herein. Dual function guidewire 210 may include a keyed configuration that allows the inner guidewire and the outer guidewire to selectively be secured together and released. For example, in at least some embodiments distal region 262 (e.g., of the inner guidewire) may include one or more keys or flanges 264. Flanges 264 may project from distal region 262 and define an outer dimension larger than then the lumen of tubular member 238 (e.g., of the outer guidewire). Tubular member 238 (e.g., of the outer guidewire) may include one or more longitudinal grooves 270, having a size that would permit flanges 264 to pass therethrough.

Flanges 264 may be disposed distally of tubular member 238. Accordingly, when in a first configuration (e.g., as shown in FIG. 14), proximal retraction of distal region 262 will be prevented by tubular member 238. In other words, the inner guidewire will not be able to be retracted out from the outer guidewire. In addition, distal pushing forces applied onto tubular member 238 will be transferred to flanges 264 and, thus, the inner guidewire. Rotating distal region 262 so that flanges 264 align with grooves 270 (e.g., as shown in FIG. 15) allows the inner guidewire to be proximally retracted.

The materials that can be used for the various components of dual function guidewire 10 (and/or other dual function guidewires disclosed herein) may include those commonly associated with medical devices. For simplicity purposes, the following discussion makes reference to dual function guidewire 10. However, this is not intended to limit the devices and methods described herein, as the discussion may be applied to other similar tubular members and/or components of tubular members or devices disclosed herein.

Dual function guidewire 10 and/or other components thereof may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or dual function guidewire 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of dual function guidewire 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of dual function guidewire 10 to achieve the same result.

In some embodiments, a degree of Magnetic Resonance Imaging (MRI) compatibility is imparted into dual function guidewire 10. For example, dual function guidewire 10, or portions thereof, may be made of a material that does not substantially distort the image and create substantial artifacts (i.e., gaps in the image). Certain ferromagnetic materials, for example, may not be suitable because they may create artifacts in an MRI image. Dual function guidewire 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In at least some embodiments, slots 48 may also define an “uneven” surface along outer guidewire 36 that may increase the ability of dual function guidewire 10 (and/or outer guidewire 36) to be imaged using an ultrasound imaging system. In some of these and in other embodiments, dual function guidewire 10 may also include other structural features that increase the echogenicity or otherwise increase the ability of dual function guidewire 10 and/or components thereof to be imaged via ultrasound.

Various embodiments of arrangements and configurations of slots are also contemplated that may be used in addition to what is described above or may be used in alternate embodiments. For simplicity purposes, the following disclosure makes reference to slots 48, and tubular member 38. However, it can be appreciated that these variations may also be utilized for other slots and/or tubular members disclosed herein. In some embodiments, at least some, if not all of slots 48 are disposed at the same or a similar angle with respect to the longitudinal axis of tubular member 38. As shown, slots 48 can be disposed at an angle that is perpendicular, or substantially perpendicular, and/or can be characterized as being disposed in a plane that is normal to the longitudinal axis of tubular member 38. However, in other embodiments, slots 48 can be disposed at an angle that is not perpendicular, and/or can be characterized as being disposed in a plane that is not normal to the longitudinal axis of tubular member 38. Additionally, a group of one or more slots 48 may be disposed at different angles relative to another group of one or more slots 48. The distribution and/or configuration of slots 48 can also include, to the extent applicable, any of those disclosed in U.S. Pat. Publication No. US 2004/0181174, the entire disclosure of which is herein incorporated by reference.

Slots 48 may be provided to enhance the flexibility of tubular member 38 while still allowing for suitable torque transmission characteristics. Slots 48 may be formed such that one or more rings and/or tube segments interconnected by one or more segments and/or beams that are formed in tubular member 38, and such tube segments and beams may include portions of tubular member 38 that remain after slots 48 are formed in the body of tubular member 38. Such an interconnected structure may act to maintain a relatively high degree of torsional stiffness, while maintaining a desired level of lateral flexibility. In some embodiments, some adjacent slots 48 can be formed such that they include portions that overlap with each other about the circumference of tubular member 38. In other embodiments, some adjacent slots 48 can be disposed such that they do not necessarily overlap with each other, but are disposed in a pattern that provides the desired degree of lateral flexibility.

Additionally, slots 48 can be arranged along the length of, or about the circumference of, tubular member 38 to achieve desired properties. For example, adjacent slots 48, or groups of slots 48, can be arranged in a symmetrical pattern, such as being disposed essentially equally on opposite sides about the circumference of tubular member 38, or can be rotated by an angle relative to each other about the axis of tubular member 38. Additionally, adjacent slots 48, or groups of slots 48, may be equally spaced along the length of tubular member 38, or can be arranged in an increasing or decreasing density pattern, or can be arranged in a non-symmetric or irregular pattern. Other characteristics, such as slot size, slot shape, and/or slot angle with respect to the longitudinal axis of tubular member 38, can also be varied along the length of tubular member 38 in order to vary the flexibility or other properties. In other embodiments, moreover, it is contemplated that the portions of the tubular member, such as a proximal section, or a distal section, or the entire tubular member 38, may not include any such slots 48.

As suggested herein, slots 48 may be formed in groups of two, three, four, five, or more slots 48, which may be located at substantially the same location along the axis of tubular member 38. Alternatively, a single slot 48 may be disposed at some or all of these locations. Within the groups of slots 48, there may be included slots 48 that are equal in size (i.e., span the same circumferential distance around tubular member 38). In some of these as well as other embodiments, at least some slots 48 in a group are unequal in size (i.e., span a different circumferential distance around tubular member 38). Longitudinally adjacent groups of slots 48 may have the same or different configurations. For example, some embodiments of tubular member 38 include slots 48 that are equal in size in a first group and then unequally sized in an adjacent group. It can be appreciated that in groups that have two slots 48 that are equal in size and are symmetrically disposed around the tube circumference, the centroid of the pair of beams (i.e., the portion of tubular member 38 remaining after slots 48 are formed therein) is coincident with the central axis of tubular member 38. Conversely, in groups that have two slots 48 that are unequal in size and whose centroids are directly opposed on the tube circumference, the centroid of the pair of beams can be offset from the central axis of tubular member 38. Some embodiments of tubular member 38 include only slot groups with centroids that are coincident with the central axis of the tubular member 38, only slot groups with centroids that are offset from the central axis of tubular member 38, or slot groups with centroids that are coincident with the central axis of tubular member 38 in a first group and offset from the central axis of tubular member 38 in another group. The amount of offset may vary depending on the depth (or length) of slots 48 and can include other suitable distances.

Slots 48 can be formed by methods such as micro-machining, saw-cutting (e.g., using a diamond grit embedded semiconductor dicing blade), electron discharge machining, grinding, milling, casting, molding, chemically etching or treating, or other known methods, and the like. In some such embodiments, the structure of the tubular member 38 is formed by cutting and/or removing portions of the tube to form slots 48. Some example embodiments of appropriate micromachining methods and other cutting methods, and structures for tubular members including slots and medical devices including tubular members are disclosed in U.S. Pat. Publication Nos. 2003/0069522 and 2004/0181174-A2; and U.S. Pat. No. 6,766,720; and U.S. Pat. No. 6,579,246, the entire disclosures of which are herein incorporated by reference. Some example embodiments of etching processes are described in U.S. Pat. No. 5,106,455, the entire disclosure of which is herein incorporated by reference. It should be noted that the methods for manufacturing dual function guidewire 10/110 may include forming slots 48 in tubular member 38 using these or other manufacturing steps.

In at least some embodiments, slots 48 may be formed in tubular member 38 using a laser cutting process. The laser cutting process may include a suitable laser and/or laser cutting apparatus. For example, the laser cutting process may utilize a fiber laser. Utilizing processes like laser cutting may be desirable for a number of reasons. For example, laser cutting processes may allow tubular member 38 to be cut into a number of different cutting patterns in a precisely controlled manner. This may include variations in the slot width, ring width, beam height and/or width, etc. Furthermore, changes to the cutting pattern can be made without the need to replace the cutting instrument (e.g., blade). This may also allow smaller tubes (e.g., having a smaller outer diameter) to be used to form tubular member 38 without being limited by a minimum cutting blade size. Consequently, tubular member 38 may be fabricated for use in neurological devices or other devices where a relatively small size may be desired.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A dual function medical device, comprising:
an inner guidewire;
an outer guidewire disposed about the inner guidewire, the outer guidewire including a tubular member having a plurality of slots formed therein;
a torque member, the torque member being configured to independently attach to the inner guidewire and the outer guidewire.

2. The dual function medical device of claim 1, wherein the inner guidewire, the outer guidewire, or both have one or more pre-formed bends formed therein.

3. The dual function medical device of claim 1, wherein the inner guidewire, the outer guidewire, or both have a distal region that is configured to buckle into a loop configuration.

4. The dual function medical device of claim 1, wherein the outer guidewire has a distal collet member.

5. The dual function medical device of claim 4, wherein the inner guidewire has a shoulder formed therein and wherein the distal collet member is configured to engage the shoulder.

6. The dual function medical device of claim 5, wherein the distal collet member is configured to shift between a first configuration wherein the distal collet member engages the shoulder and a deflected configuration.

7. The dual function medical device of claim 1, wherein the inner guidewire has an outer diameter of 0.010-0.025 inches and wherein the outer guidewire has an outer diameter of 0.025-0.040 inches.

8. The dual function medical device of claim 1, wherein the torque member includes a first locking member configured to be secured to the inner guidewire and a second locking member configured to be secured to the outer guidewire.

9. The dual function medical device of claim 1, wherein the inner guidewire has a distal region having one or more flanges, and wherein the outer guidewire has one or more longitudinal grooves formed therein.

10. A method for accessing a region of the biliary tree, the pancreatic tract, or both, the method comprising:
providing a dual function guidewire, the dual function guidewire including:
an inner guidewire, and
an outer guidewire disposed about the inner guidewire, the outer guidewire including a tubular member having a plurality of slots formed therein;
advancing the dual function guidewire through a body lumen to a position adjacent to an area of interest; and
either advancing a first medical device over the outer guidewire to the area of interest or removing the outer guidewire from the inner guidewire and advancing a second medical device over the inner guidewire to the area of interest.

11. The method of claim 10, wherein advancing the dual function guidewire through a body lumen to a position adjacent to an area of interest includes positioning the inner guidewire so that the inner guidewire extends distally from a distal end of the outer guidewire.

12. The method of claim 10, wherein either advancing a first medical device over the outer guidewire to the area of interest or removing the outer guidewire from the inner guidewire and advancing a second medical device over the inner guidewire to the area of interest includes advancing the first medical device of the outer guidewire to the area of interest.

13. The method of claim 10, wherein either advancing a first medical device over the outer guidewire to the area of interest or removing the outer guidewire from the inner guidewire and advancing a second medical device over the inner guidewire to the area of interest includes removing the outer guidewire from the inner guidewire and advancing the second medical device over the inner guidewire to the area of interest.

14. The method of claim 10, wherein the inner guidewire has one or more pre-formed bends formed therein.

15. The method of claim 10, wherein the inner guidewire has a distal region that is configured to buckle into a loop configuration.

16. The method of claim 10, wherein the outer guidewire has a distal collet member, wherein the inner guidewire has a shoulder formed therein and wherein the distal collect member is configured to engage the shoulder, and wherein the distal collet member is configured to shift between a first configuration wherein the distal collet member engages the shoulder and a deflected configuration.

17. The method of claim 10, wherein a torque member is coupled to the dual function guidewire, the torque member including a first locking member configured to be secured to the inner guidewire and a second locking member configured to be secured to the outer guidewire.

18. The method of claim 10, wherein the area of interest includes a bile duct.

19. The method of claim 10, wherein the area of interest includes a pancreatic duct.

20. A dual function guidewire for accessing a body lumen along the biliary tree of a patient, the dual function guidewire comprising:
an inner guidewire, the inner guidewire having first outer diameter that is configured to guide a first medical device to an area of interest;
an outer guidewire disposed about the inner guidewire, the outer guidewire having a second outer diameter that is configured to guide a second medical device to the area of interest;
wherein the inner guidewire has a pre-formed bend formed therein;
wherein the outer guidewire includes a tubular member having a plurality of slots formed therein; and
wherein the first outer diameter closely approximates an inner diameter of the outer guidewire.
